(19) United States
(12) Patent Application Publication
(10) Pub. No.: US 2019/0142308 A1
(43) Pub. Date: May 16, 2019

(54) BABY BREATHING MONITOR AND SYSTEM THEREOF

(71) Applicant: Crystal Washington, Fairburn, GA (US)

(72) Inventor: Crystal Washington, Fairburn, GA (US)

(21) Appl. No.: 15/810,663

(22) Filed: Nov. 13, 2017

(51) Int. Cl. A61B 5/113; A61B 5/0205; A61B 5/00

(52) U.S. Cl. CPC A61B 5/1135; A61B 5/02055; A61B 5/742; A61B 5/08; A61B 5/6804; A61B 5/0004; A61B 5/746

(57) ABSTRACT

A baby breathing monitor system, including a baby breathing sensor to be installed within a shirt to monitor a breathing of a baby, and a baby breathing monitor to communicate with the baby breathing sensor and to alert a user when the baby breathing sensor detects that the breathing of the baby has ceased.

### BACKGROUND OF THE INVENTION

### 1. Field of the Invention

The present general inventive concept relates generally to a baby breathing monitor, and specifically, a baby breathing monitor and system thereof.

### 2. Description of the Related Art

Sudden infant death syndrome (SIDS) is the sudden unexplained death of a child less than one year of age. Although SIDS remains often unexplained, some professionals believe that infants simply cease breathing due to asphyxiation, or even cardiac arrest.

As such, there is a need for a baby monitor that monitors an infant's breathing, in order to prevent SIDS for infants in the future.

There is also a need for a baby monitor that alerts a user of different needs of an infant.

### SUMMARY

The present general inventive concept provides a baby breathing monitor and system thereof.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a baby breathing monitor system, including a baby breathing sensor to be installed within a shirt to monitor a breathing of a baby, and a baby breathing monitor to communicate with the baby breathing sensor and to alert a user when the baby breathing sensor detects that the breathing of the baby has ceased.

The baby breathing sensor may include at least one motion sensor disposed on a rear portion of the baby breathing sensor to detect a motion of the baby, a processor to receive and interpret the motion detection from the at least one sensor, and a transmitter to transmit a signal from the baby breathing sensor to the baby breathing monitor in response to the processor interpreting that the motion of the baby has ceased.

The baby breathing monitor may include a display unit to display an alert message to alert the user that the motion of the baby has ceased.

The display unit may include a touch screen to allow the user to input information regarding at least one reminder into the baby breathing monitor, the at least one reminder including at least one of a birthdate of the baby, feeding times of the baby, doctor appointments of the baby, and vaccination dates of the baby.

The display unit may display a reminder message to alert the user of the at least one reminder.

The baby breathing sensor may include at least one temperature sensor to sense a temperature of the baby.

The baby breathing sensor may include at least one vibration sensor to sense a heartbeat of the baby.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1A illustrates a baby breathing monitor system, according to an exemplary embodiment of the present general inventive concept;

FIG. 1B illustrates a rear view of a baby breathing sensor, according to an exemplary embodiment of the present general inventive concept;

FIG. 2 illustrates a baby breathing sensor installed within a shirt, according to an exemplary embodiment of the present general inventive concept;

FIG. 3 illustrates a baby breathing sensor installed within a shirt, according to another exemplary embodiment of the present general inventive concept;

FIG. 4 illustrates a baby breathing sensor installed within a shirt, according to another exemplary embodiment of the present general inventive concept; and

FIGS. 5A and 5B illustrate a baby breathing monitor embodied as a smart watch, according to various embodiments of the present general inventive concept.

### DETAILED DESCRIPTION OF THE INVENTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

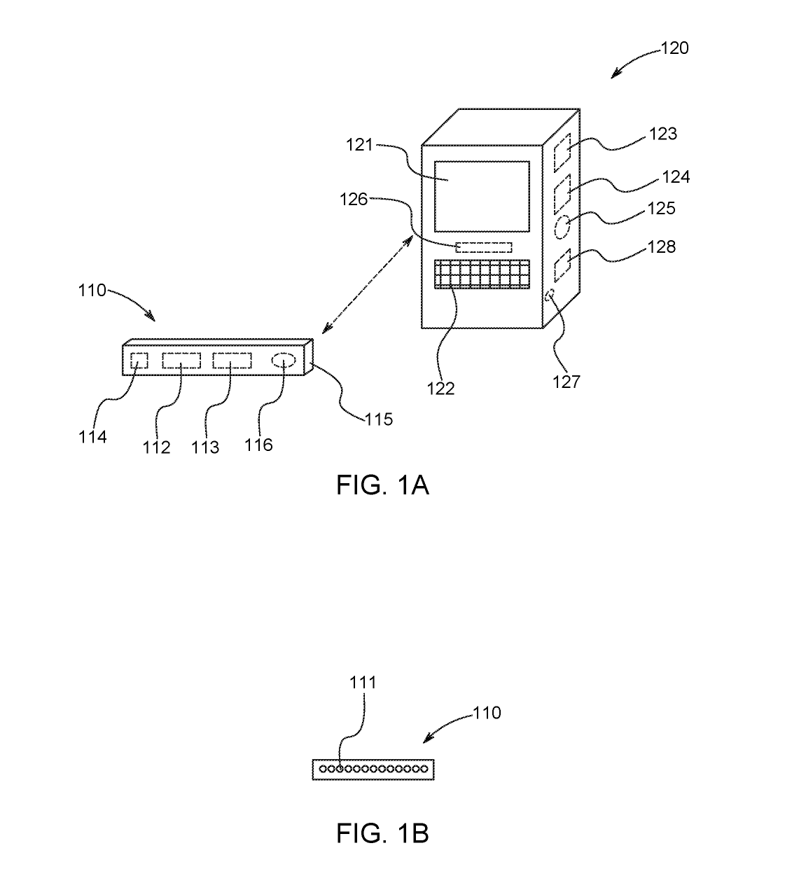

FIG. 1A illustrates a baby breathing monitor system 100, according to an exemplary embodiment of the present general inventive concept.

Referring to FIG. 1A, the baby breathing monitor system 100 may include a baby breathing sensor 110 and a baby breathing monitor 120.

The baby breathing sensor 110 may be constructed from ultra-lightweight material, in order to not obstruct a baby's breathing when a baby had the baby breathing sensor 110 placed upon a chest of the baby.

The baby breathing sensor 110 may communicate with the baby breathing monitor 120 using BLUETOOTH, WIFI, satellite, 3G, 4G, near-field communication, radio frequency communication, radio waves, or any other form of communication known to one of ordinary skill in the art.

The baby breathing sensor 110 may be placed upon a chest of a baby, either inside a shirt of the baby, or directly against the chest of the baby. In other words, the baby breathing sensor 110 is designed to be worn by the baby on a chest area thereof.

FIG. 1B illustrates a rear view of the baby breathing sensor 110, according to an exemplary embodiment of the present general inventive concept

Referring to FIGS. 1A and 1B, the baby breathing sensor 110 may include at least one sensor 111 disposed on a rear portion thereof, which may sense (i.e., detect) whether a baby wearing the baby breathing sensor 110 is still breathing, and a processor 112 to receive and interpret the detection from the at least one sensor 111.

The at least one sensor 111 may be a motion sensor, a vibration sensor, a tactile sensor, an accelerometer-type sensor, a pressure sensor, and a temperature sensor, but is not limited thereto.

The baby breathing sensor 110 may also include a transmitter 113, which may transmit a signal from the baby breathing sensor 110 to the baby breathing monitor 120.

More specifically, if the at least one sensor 111 detects that the baby has stopped breathing, then the processor 112 controls the transmitter 113 to send a signal to the baby breathing monitor 120.

As such, for example, if the at least one sensor 111 is embodied as a motion sensor and the baby breathing sensor is placed on the chest of the baby, then a ceasing of a breathing of the baby will cause the at least one sensor to detect that motion of the at least one sensor 111 has ceased. Accordingly, if the baby stops breathing and the baby breathing sensor 110 stops moving, the at least one sensor 111 sends a CEASED MOTION signal to the processor, which controls at least one speaker 114 to emit a noise and also controls the transmitter 113 to send a signal to the baby breathing monitor 120 to alert the user that the baby has stopped breathing.

The baby breathing sensor 110 may include a USB port 115 connected to a battery 116 to allow the battery 116 of the baby breathing sensor 110 to be easily recharged.

The baby breathing monitor 120 may include a display unit 121, a keypad 122, a processor 123, a transmitter 124, a receiver 125, a speaker 126, a USB port 127, and a battery 128.

The display unit 121 may be a touch screen, an LCD screen, a plasma screen, an LED screen, or any other type of display unit known to one of ordinary skill in the art.

The display unit 121 may allow a user to input information using the keypad 122 (or alternatively, the touchpad if the display unit 121 is embodied as such) regarding reminders into the baby breathing monitor, such as a birthdate of the baby, important scheduling information such as feeding times/doctor appointments/vaccination dates, and other optional important reminders. As such, when a reminder is triggered based on a particular date and/or time, the speaker 126 may emit an alert sound, and the display unit 121 may display an alert message regarding the reminder. Also, the display unit 121 may allow the user to access various web pages on the Internet.

The processor 123 may store the input information and/or the reminders in a storage unit 123a, and may control the display unit 121 to display the alert message and the speaker 126 to emit the alert sound.

The processor 123 may also control the transmitter 124 to send signals to the baby breathing sensor 110, and may also process signals received from the baby breathing sensor 110 by the receiver 125. In other words, the processor 123 may include software to allow for communication between the baby breathing monitor 120 and the baby breathing sensor 110.

The USB port 127 may allow the battery 128 to be recharged. Alternatively, the baby breathing monitor 120 may be plugged into an electrical socket.

As another example, if the baby breathing sensor 110 is embodied as a vibration sensor, then a heartbeat of the baby may also be monitored to alert the user if the heartbeat of the baby ceases.

As yet another example, if the baby breathing sensor 110 is embodied as a temperature sensor, then a body temperature of the baby may also be monitored on the display unit 121.

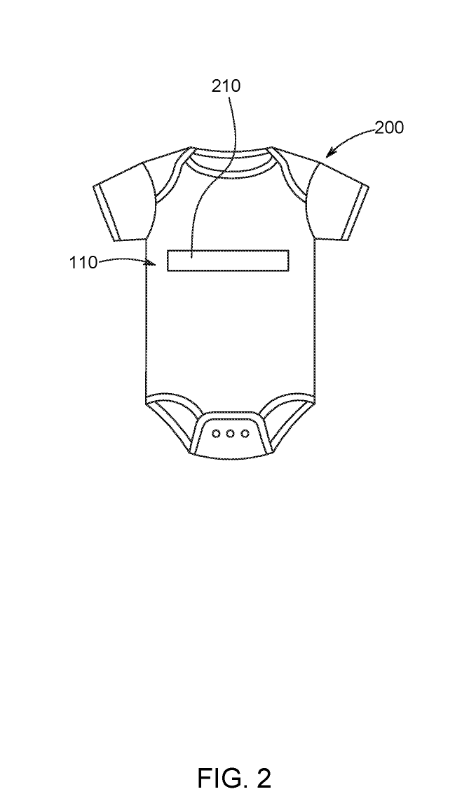

FIG. 2 illustrates the baby breathing sensor 110 installed within a shirt 200, according to an exemplary embodiment of the present general inventive concept.

The shirt 200 may include a pocket 210 to allow the baby breathing sensor 110 to be inserted therein.

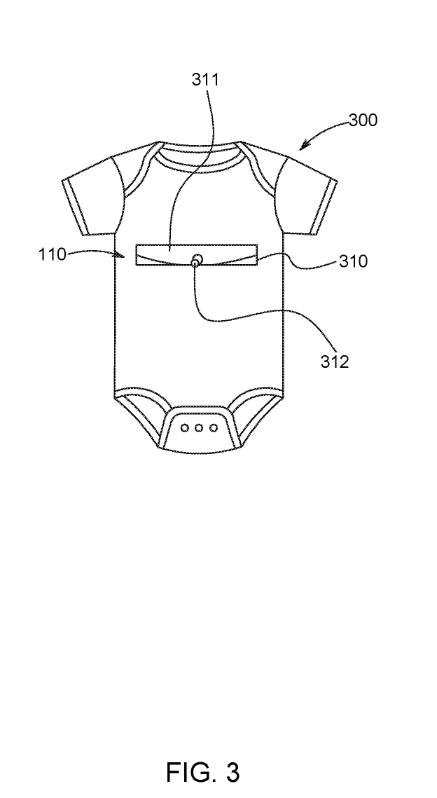

FIG. 3 illustrates the baby breathing sensor 110 installed within a shirt 300, according to another exemplary embodiment of the present general inventive concept.

The shirt 300 may include a pocket 310 to allow the baby breathing sensor 110 to be inserted therein.

The pocket 310 may include a pocket cover 311 and a closing device 312 to secure the baby breathing sensor 110 inside the pocket 310. The closing device 312 may be a snap, a zipper, VELCRO, a magnet, or any other type of closing device known to one of ordinary skill in the art.

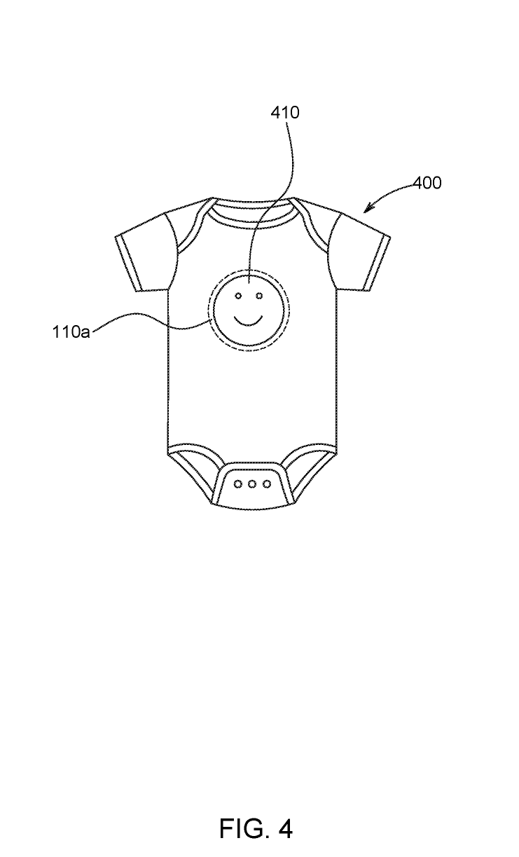

FIG. 4 illustrates a baby breathing sensor 110a installed within a shirt 400, according to another exemplary embodiment of the present general inventive concept.

The baby breathing sensor 110a may have a shape to be installed behind a patch 410 having a corresponding shape, as illustrated in FIG. 4.

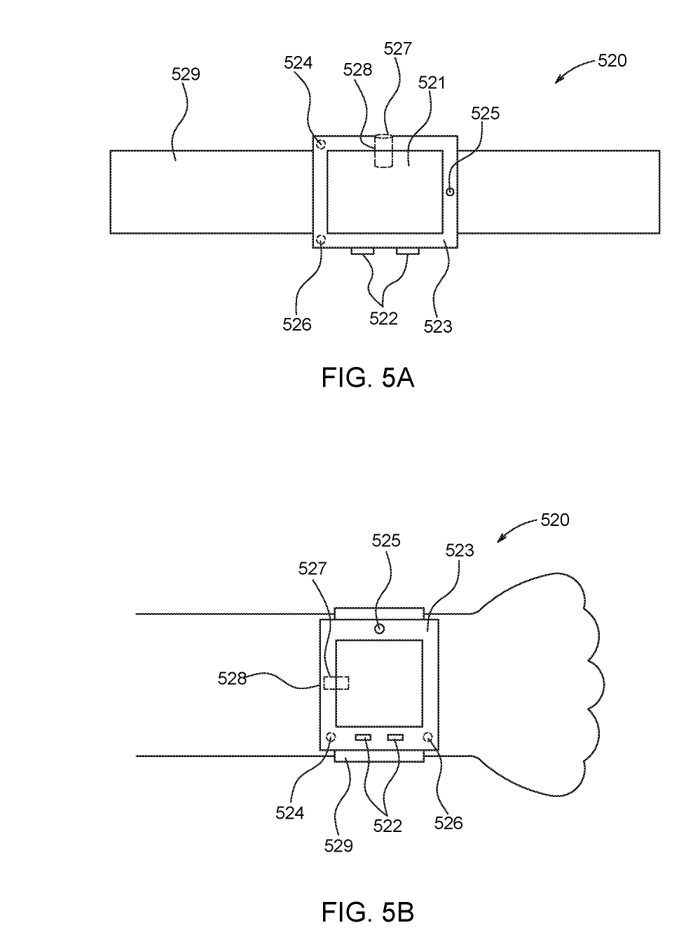

FIGS. 5A and 5B illustrate a baby breathing monitor 520 embodied as a smart watch, according to various embodiments of the present general inventive concept.

Referring to FIGS. 5A and 5B, the baby breathing monitor 520 may include a display unit 521, at least one button 522, a processor 523, a speaker 524, a camera 525, a microphone 526, a USB port 527, a battery 528, and a strap 529.

The display unit 521 may be a touch screen, an LCD screen, a plasma screen, an LED screen, or any other type of display unit known to one of ordinary skill in the art.

When the display unit 521 is embodied as the touch screen, the display unit 521 may allow a user to access various web pages on the Internet, input information thereupon regarding reminders into the baby breathing monitor, such as a birthdate of the baby, important scheduling information such as feeding times/doctor appointments/vaccination dates, and other optional important reminders. As such, when a reminder is triggered based on a particular date and/or time, the speaker 524 may emit an alert sound, and the display unit 521 may display an alert message regarding the reminder.

The at least one button 522 may be disposed on a side of the baby breathing monitor 520 as illustrated in FIG. 5A, or alternatively, on a front face of the baby breathing monitor 520 as illustrated in FIG. 5B.

The user may use the at least one button 522 to input information in a manner same as the display unit 521.

The processor 523 may store the input information and/or the reminders in a storage unit 523a, and may control the display unit 521 to display the alert message and the speaker 524 to emit the alert sound.

The processor 523 may have software running thereon to allow for communication with the baby breathing sensor 110, and may also process signals received from the baby breathing sensor 110. In other words, the processor 523 may include software to allow for communication between the baby breathing monitor 520 and the baby breathing sensor 110.

The camera 525 may allow a user to take pictures and/or video chat.

The microphone 526 may be used to allow the user to communicate with other baby breathing monitors 520, or to allow audio to be transmitted during a video chat.

The speaker 524 may also allow audio to be heard during a video chat or during a communication from another baby breathing monitor.

The USB port 527 may allow the battery 528 to be recharged.

The strap 529 may allow the user to wear the baby breathing monitor 520.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A baby breathing monitor system, comprising:
a baby breathing sensor to be installed within a shirt to monitor a breathing of a baby; and
a baby breathing monitor to communicate with the baby breathing sensor and to alert a user when the baby breathing sensor detects that the breathing of the baby has ceased.

2. The baby breathing monitor system of claim 1, wherein the baby breathing sensor comprises:
at least one motion sensor disposed on a rear portion of the baby breathing sensor to detect a motion of the baby;
a processor to receive and interpret the motion detection from the at least one sensor; and
a transmitter to transmit a signal from the baby breathing sensor to the baby breathing monitor in response to the processor interpreting that the motion of the baby has ceased.

3. The baby breathing monitor system of claim 2, wherein the baby breathing monitor comprises:
a display unit to display an alert message to alert the user that the motion of the baby has ceased.

4. The baby breathing monitor system of claim 3, wherein the display unit comprises:
a touch screen to allow the user to input information regarding at least one reminder into the baby breathing monitor, the at least one reminder comprising at least one of a birthdate of the baby, feeding times of the baby, doctor appointments of the baby, and vaccination dates of the baby.

5. The baby breathing monitor of claim 4, wherein the display unit displays a reminder message to alert the user of the at least one reminder.

6. The baby breathing monitor system of claim 1, wherein the baby breathing sensor comprises:
at least one temperature sensor to sense a temperature of the baby.

7. The baby breathing monitor system of claim 1, wherein the baby breathing sensor comprises:
at least one vibration sensor to sense a heartbeat of the baby.
